(19) United States
(12) Patent Application Publication
(10) Pub. No.: US 2025/0042864 A1
(43) Pub. Date: Feb. 6, 2025

(54) SOLID STATE FORM OF TAFAMIDIS

(71) Applicant: TEVA PHARMACEUTICALS INTERNATIONAL GMBH, Jona (CH)

(72) Inventors: Sanja Matecic Musanic, Zagreb (HR); Valentina Travancic, Zagreb (HR); Dubravka Pavlicic, Zagreb (HR); Marina Ratkaj, Zagreb (HR)

(21) Appl. No.: 18/710,701

(22) PCT Filed: Nov. 17, 2022
(86) PCT No.: PCT/US2022/050182

§ 371 Date: May 16, 2024

Related U.S. Application Data
(60) Provisional application No. 63/280,259, filed on Nov. 17, 2021.

(51) Int. Cl. C07D 263/58; A61K 31/423

(52) U.S. Cl. CPC C07D 263/58; A61K 31/423

(57) ABSTRACT

The present disclosure relates to a solid state form of Tafamidis, processes for preparation thereof and pharmaceutical compositions thereof.

### TECHNICAL FIELD

The present disclosure relates to a solid state form of Tafamidis, processes for preparation thereof and pharmaceutical compositions thereof.

### BACKGROUND

Tafamidis has the chemical name 2-(3,5-dichlorophenyl)-1,3-benzoxazole-6-carboxylic acid. Tafamidis has the following chemical structure:

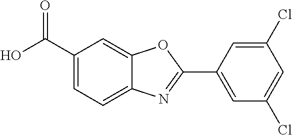 [SMILES: O=C(O)c1ccc2nc(-c3cc(Cl)cc(Cl)c3)oc2c1]

Tafamidis meglumine has the chemical name 2-(3,5-dichlorophenyl)-1,3-benzoxazole-6-carboxylic acid mono (1-deoxy-1-methylamino-D-glucitol). Tafamidis meglumine has the following chemical structure:

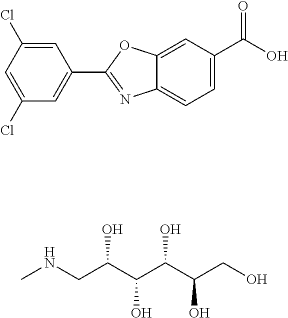 [SMILES: O=C(O)c1ccc2nc(-c3cc(Cl)cc(Cl)c3)oc2c1 ; CNC[C@H](O)[C@@H](O)[C@H](O)[C@H](O)CO]

VYNDAQEL (tafamidis meglumine) and VYNDAMAX (tafamidis), are both capsules for oral administration and contain tafamidis as the active moiety. The U.S. Food and Drug Administration (“FDA”) has approved VYNDAQEL and VYNDAMAX for the treatment of cardiomyopathy of wild type or hereditary transthyretin-mediated amyloidosis in adults to reduce cardiovascular mortality and cardiovascular-related hospitalization. EMA has approved VYNDAQEL for the treatment of transthyretin amyloidosis in adult patients with stage 1 symptomatic polyneuropathy to delay peripheral neurologic impairment.

Tafamidis is known from U.S. Pat. No. 7,214,695.

Solid state forms of tafamidis meglumine are known from U.S. Pat. No. 9,249,112, International Publication Nos. WO2017190682 and WO2019175263. Solid state forms of tafamidis are known from U.S. Pat. No. 9,770,441, International Publication Nos. WO2020232325 and WO2021001858. International Publication No. WO2021093809 discloses tafamidis cocrystals.

Polymorphism, the occurrence of different crystal forms, is a property of some molecules and molecular complexes. A single compound, like tafamidis, may give rise to a variety of polymorphs having distinct crystal structures and physical properties like melting point, thermal behaviors (e.g., measured by thermogravimetric analysis—“TGA”, or differential scanning calorimetry—“DSC”), powder X-ray diffraction (PXRD) pattern, infrared absorption fingerprint, Raman absorption fingerprint, and solid state (¹³C-) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, improving the dissolution profile, or improving stability (polymorph as well as chemical stability) and shelf-life. These variations in the properties of different salts and solid state forms may also provide improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to use variations in the properties and characteristics of a solid active pharmaceutical ingredient for providing an improved product.

Discovering new salts, solid state forms, and solvates of a pharmaceutical product can provide materials having desirable processing properties, such as ease of handling, ease of processing, storage stability, and ease of purification, or as desirable intermediate crystal forms that facilitate conversion to other salts or polymorphic forms. New salts, polymorphic forms and solvates of a pharmaceutically useful compound can also provide an opportunity to improve the performance characteristics of a pharmaceutical product (dissolution profile, bioavailability, etc.). It enlarges the repertoire of materials that a formulation scientist has available for formulation optimization, for example by providing a product with different properties, e.g., a different crystal habit, higher crystallinity, or polymorphic stability, or solubility, flowability, which may offer better processing or handling characteristics, improved dissolution profile, or improved shelf-life either in the drug product, during formulation processes, or in the final pharmaceutical formulation/dosage form.

For at least these reasons, crystalline forms (including solvated forms) of tafamidis having desirable properties remain desirable.

### SUMMARY

The present disclosure relates to a solid state form of tafamidis thereof, to processes for preparation thereof, and to pharmaceutical compositions including the solid state form.

The present disclosure also provides uses of the solid state form of tafamidis as described in any embodiment or aspect herein for preparing other solid state forms of tafamidis, tafamidis salts and solid state forms thereof.

In another embodiment, the present disclosure encompasses the solid state form of tafamidis as described in any embodiment or aspect herein for use as a medicament, in embodiments for the treatment of transthyretin-mediated amyloidosis.

In another embodiment, the present disclosure encompasses methods for treating transthyretin-mediated amyloidosis with the use of the herein disclosed solid state form of tafamidis.

In a further embodiment, the present disclosure further provides the use of the solid state form of tafamidis described according to any embodiment or aspect herein, for the preparation of a pharmaceutical composition or a pharmaceutical formulation of tafamidis, wherein the tafamidis in the pharmaceutical composition or formulation is in a solid form, wherein the solid form may be any crystalline form or an amorphous form.

The present disclosure further provides pharmaceutical compositions including the solid state form of tafamidis according to the present disclosure.

In yet another embodiment, the present disclosure encompasses pharmaceutical formulations including the solid state form of tafamidis as described in any aspect or embodiment herein, and at least one pharmaceutically acceptable excipient, in embodiments for oral administration in dosage forms such as tablets, capsules, etc.

The present disclosure encompasses processes to prepare said pharmaceutical formulations of tafamidis by combining at least the solid state form of tafamidis as described in any aspect or embodiment of the present disclosure, and at least one pharmaceutically acceptable excipient.

The solid state form as defined herein, as well as the pharmaceutical compositions or formulations of the solid state form of tafamidis, can be used as medicaments, in embodiments for the treatment of transthyretin-mediated amyloidosis.

The present disclosure also provides methods of treating transthyretin-mediated amyloidosis, by administering a therapeutically effective amount of the solid state form of tafamidis of any aspect or embodiment of the present disclosure, or at least one of the above pharmaceutical compositions or formulations, to a subject suffering from transthyretin-mediated amyloidosis, or otherwise in need of the treatment.

The present disclosure also provides use of the solid state form of tafamidis of any aspect or embodiment of the present disclosure, or at least one of the above pharmaceutical compositions or formulations, for the manufacture of a medicament for treating transthyretin-mediated amyloidosis.

### BRIEF DESCRIPTION OF THE FIGURES

FIG. 1 shows a powder X-ray diffraction pattern (“powder XRD” or “PXRD” or “XRPD”) of Form Va of tafamidis.

FIG. 2 shows a XRPD pattern of tafamidis form V (methanol solvate).

FIG. 3 shows a XRPD pattern of tafamidis form V (anhydrous).

FIG. 4 shows ¹³C NMR of anhydrous form V of tafamidis.

FIG. 5 shows XRPD patterns of tafamidis Form 4 before and after 2 hours slurry in PEG 400 at room temperature.

FIG. 6 shows XRPD patterns of tafamidis Form Va before and after 7 days slurry in PEG 400 at room temperature.

FIG. 7 shows a ¹³C NMR spectrum of Form Va of tafamidis.

### DETAILED DESCRIPTION

The present disclosure relates to a solid state form of tafamidis, in embodiments to crystalline forms of tafamidis, processes for preparation thereof, and pharmaceutical compositions including the solid state form.

The solid state form of tafamidis according to the present disclosure may have advantageous properties selected from at least one of: chemical or polymorphic purity, flowability, solubility, dissolution rate, bioavailability, morphology or crystal habit, stability—such as chemical stability as well as thermal and mechanical stability with respect to polymorphic conversion, stability towards dehydration and/or storage stability, a lower degree of hygroscopicity, low content of residual solvents, and advantageous processing and handling characteristics such as compressibility, or bulk density. The advantageous properties may provide better processing or handling characteristics, improved dissolution profile, or improved shelf-life, either in the drug product, during formulation processes, or in the final pharmaceutical formulation/dosage form.

In particular, the Form Va of the present disclosure has been found to have advantageous morphology and a small particle size, compared to, e.g. Form 1 as disclosed in U.S. Pat. No. 9,770,441. Thus, compared with the long fibre- to needle-like morphology of Form 1 having a particle size of up to 100 μm, crystalline Form Va of the present disclosure has much shorter needle/rod-like particles and are significantly smaller and more uniform in size distribution (particle size of about 5-10 μm). Tafamidis has a low aqueous solubility, and hence particles having a smaller size and more uniform particle size distribution are highly advantageous for processing into a dosage form. Thus, Form 1 tafamidis requires particle size reduction (e.g. by micronization) in order to achieve a desirable particle size distribution and uniformity and to increase dissolution. Such processing is not required for Form Va, thereby reducing processing time and costs. Moreover, the fibre-like morphology of Form 1 is associated with a higher propensity for the particles to stick together, resulting in poor flowability.

Similarly, Form 6 as disclosed in International Publication No. WO 2016/038500 has large (>100 μm) agglomerated particles with a very wide particle size distribution, which are highly undesirable for formulation processing.

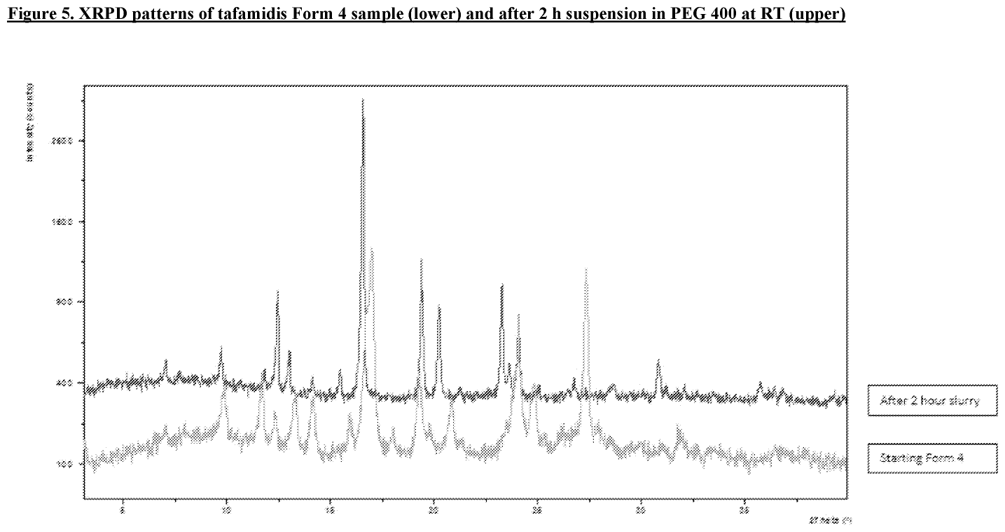

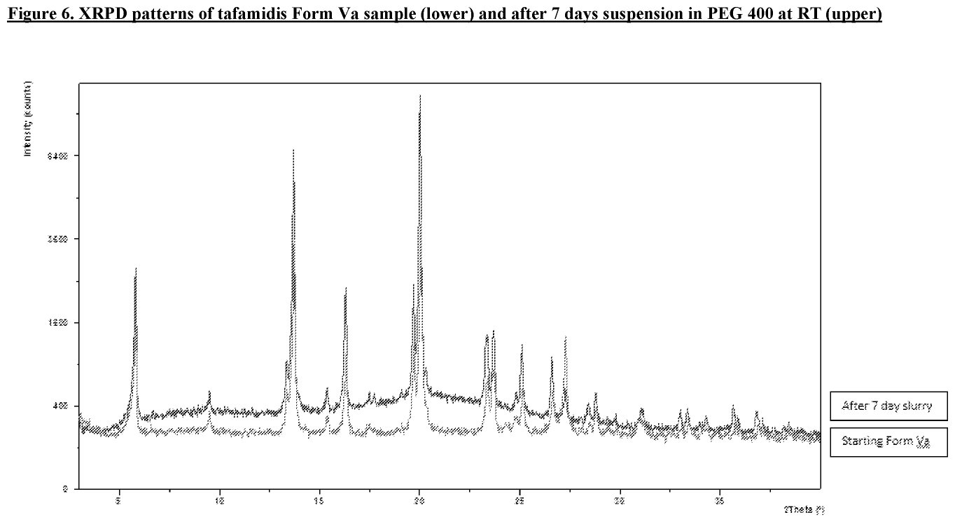

The form Va of the present disclosure is also stable to suspension in PEG, which is typically used to formulate tafamidis as a soft gel capsule. Form 4 typically converts to Form 1 after 2 hours (FIG. 5). Form Va of the present disclosure is stable for at least 7 days (FIG. 6). Form Va also exhibits a good water solubility, for example compared with Form V as disclosed in International Publication No. WO 2020/232325 (2.21 μg/ml vs 1.47 μg/ml respectively).

A crystal form may be referred to herein as being characterized by graphical data “as depicted in” a Figure. Such data include, for example, powder X-ray diffractograms and solid state NMR spectra. As is well-known in the art, the graphical data potentially provides additional technical information to further define the respective solid state form (a so-called “fingerprint”) which can not necessarily be described by reference to numerical values or peak positions alone. In any event, the skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of tafamidis and salts thereof referred to herein as being characterized by graphical data “as depicted in” a Figure will thus be understood to include any crystal forms of the tafamidis and salts thereof, characterized with the graphical data having such small variations, as are well known to the skilled person, in comparison with the Figure.

A solid state form (or polymorph) may be referred to herein as polymorphically pure or substantially free of any other solid state (or polymorphic) forms. As used herein in this context, the expression “substantially free of any other forms” will be understood to mean that the solid state form contains about 20% or less, about 10% or less, about 5% or less, about 2% or less, about 1% or less, or about 0% of any other forms of the subject compound as measured, for example, by PXRD. Thus, solid state forms of tafamidis and tafamidis salts, described herein as substantially free of any other solid state forms, would be understood to contain greater than about 80% (w/w), greater than about 90% (w/w), greater than about 95% (w/w), greater than about 98% (w/w), greater than about 99% (w/w), or about 100% (w/w) of the subject solid state form of tafamidis and/or tafamidis salts. Accordingly, in some embodiments of the disclosure, the described solid state forms of tafamidis and/or tafamidis salts may contain from about 1% to about 20% (w/w), from about 5% to about 20% (w/w), or from about 5% to about 10% (w/w) of one or more other solid state forms of the same tafamidis and/or tafamidis salts.

As used herein, unless stated otherwise, PXRD peaks reported herein are measured using CuK _{α} radiation, λ=1.541874 Å. Also, as used herein, unless stated otherwise, PXRD measurements are taken at 25° C.±3° C.

As used herein, the term “isolated” in reference to the solid state form of tafamidis of the present disclosure corresponds to a solid state form of tafamidis which is physically separated from the reaction mixture in which it is formed.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C. A process or step may be referred to herein as being carried out “overnight.” This refers to a time interval, e.g., for the process or step, that spans the time during the night, when that process or step may not be actively observed. This time interval is from about 8 to about 20 hours, or about 10 to about 18 hours, in embodiments about 16 hours.

The term “solvate”, as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

The crystal hydrate indicated by water analysis by Karl Fischer (KF) titration or by TGA analysis of the product is believed to have been produced as a result of water introduced from the atmosphere in which this material was processed, or by traces of water present in the solvents that were in contact with the material, or a combination of these factors.

The amount of solvent employed in a chemical process, e.g., a reaction or a crystallization, may be referred to herein as a number of “volumes” or “vol” or “V.” For example, a material may be referred to as being suspended (or dissolved) in 10 volumes (or 10 vol or 10V) of a solvent. In this context, this expression would be understood to mean milliliters of the solvent per gram of the material being suspended (or dissolved), such that suspending (or dissolving) 5 grams of a material in 10 volumes of a solvent means that the solvent is used in an amount of 10 milliliters of the solvent per gram of the material that is being suspended (or dissolved) or, in this example, 50 mL of the solvent. In another context, the term “v/v” may be used to indicate the number of volumes of a solvent that are added to a liquid mixture based on the volume of that mixture. For example, adding methyl tert-butyl ether (MTBE) (1.5 v/v) to a 100 ml reaction mixture would indicate that 150 mL of MTBE was added.

As used herein, the term “reduced pressure” refers to a pressure of about 10 mbar to about 50 mbar.

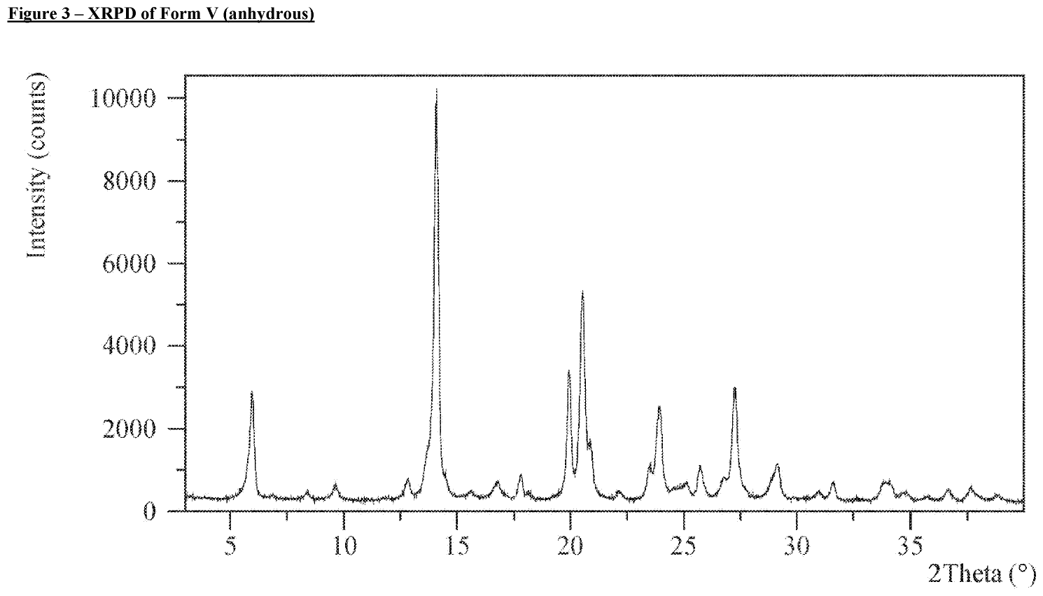

As used herein, crystalline Form V of tafamidis refers to a crystalline form as described in International Publication No. WO2020232325. In particular, crystalline Form V of tafamidis as used herein may be characterized by a PXRD pattern having peaks at 6.0, 19.9, 20.6, 23.9 and 29.2 degrees 2-theta±0.2 degrees 2-theta. Crystalline Form V of tafamidis may be alternatively characterized by a PXRD pattern having peaks at 6.0, 19.9, 20.6, 23.9 and 29.2 degrees 2-theta±0.2 degrees 2-theta, and also having one, two, three or four additional peaks at 17.8, 25.8, 27.3 and 31.1 degrees 2-theta±0.2 degrees 2-theta, and particularly by a PXRD pattern having peaks at 6.0, 17.8, 19.9, 20.6, 23.9, 25.8, 27.3, 29.2 and 31.1 degrees 2-theta±0.2 degrees 2-theta. Alternatively, crystalline Form V of tafamidis may be characterized by a PXRD pattern substantially as depicted in FIG. 3. The crystalline Form V of tafamidis is preferably an anhydrous form.

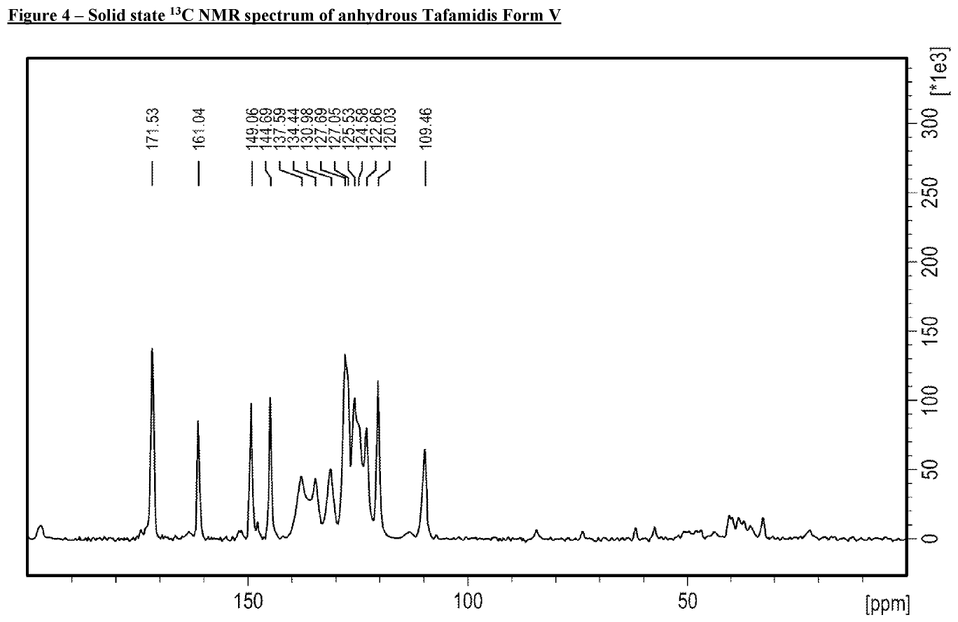

Alternatively, or additionally, Form V of tafamidis may alternatively or additionally be characterized by a solid state ¹³C NMR spectrum having peaks at 171.5, 161.0, 149.1, 144.7, 131.0±0.2 ppm and/or a solid state ¹³C NMR spectrum having the following chemical shift absolute differences from a reference peak at 109.5 ppm±0.2 ppm of 62.1, 51.6, 39.6, 35.2, 21.5±0.1 ppm. Form V of tafamidis may alternatively or additionally be characterized by a solid state ¹³C NMR spectrum substantially as depicted in FIG. 4. Crystalline form V of tafamidis as defined according to any of the above, and preferably anhydrous crystalline form V of tafamidis as defined according to any of the above, may be used as a starting material for preparing Form Va of tafamidis.

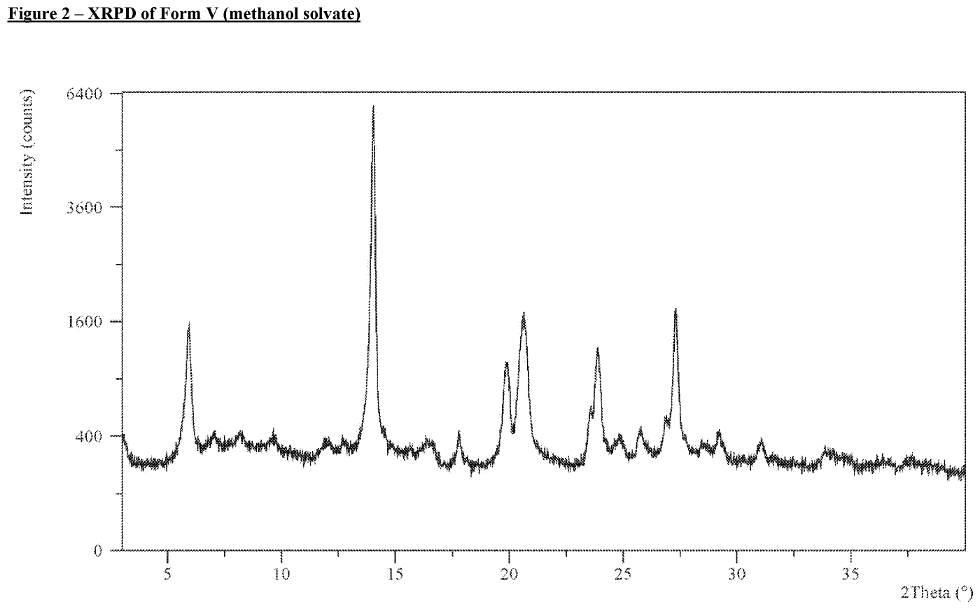

As used herein tafamidis methanol solvate is as defined in WO2020/232325 and may be prepared for example, according to Example 15 of WO2020/232325. Tafamidis methanol solvate may be characterized by a PXRD pattern substantially as depicted in FIG. 2. Anhydrous tafamidis may be prepared by the processes described herein, or may be prepared by drying Tafamidis methanol solvate in a vacuum dryer, at elevated temperature.

The present disclosure provides a crystalline form of tafamidis, designated form Va, which is characterized by an X-ray powder diffraction having peaks at 13.3, 16.3 and 19.7 degrees two-theta±0.2 degrees two-theta. The crystalline form of tafamidis may be further characterized by an X-ray powder diffraction pattern having any one, two, three, four or five additional peaks selected from: 5.8, 9.5, 13.7, 20.0, and 28.8 degrees two-theta±0.2 degrees two-theta. In embodiments the crystalline form of tafamidis may be characterized by an X-ray powder diffraction pattern having peaks at 5.8, 9.5, 13.3, 13.7, 16.3, 19.7, 20.0, and 28.8 degrees two-theta±0.2 degrees two-theta.

Alternatively, the crystalline form Va of tafamidis may be characterized by an X-ray powder diffraction pattern having peaks at 5.8, 9.5, 13.3, 13.7, 16.3, 19.7, 20.0, and 28.8 degrees two-theta±0.2 degrees two-theta, and further characterized by an X-ray powder diffraction pattern having one, two, three, four, five, or six additional peaks selected from: 23.3, 23.4, 23.7, 25.1, and 26.6 degrees two-theta±0.2 degrees two-theta. In embodiments, crystalline Form Va of tafamidis may be characterized by an X-ray powder diffraction pattern having peaks at 5.8, 9.5, 13.3, 13.7, 16.3, 19.7, 20.0, 23.3, 23.4, 23.7, 25.1, and 26.6 degrees two-theta±0.2 degrees two-theta.

Crystalline form of tafamidis, may be alternatively characterized by an X-ray powder diffraction pattern having peaks at 13.3, 16.3 and 19.7 degrees two-theta±0.1 degrees two-theta. The crystalline form of tafamidis may be further characterized by an X-ray powder diffraction pattern having any one, two, three, four or five additional peaks selected from: 5.8, 9.5, 13.7, 20.0, and 28.8 degrees two-theta±0.1 degrees two-theta. In embodiments the crystalline form of tafamidis may be characterized by an X-ray powder diffraction pattern having peaks at 5.8, 9.5, 13.3, 13.7, 16.3, 19.7, 20.0, and 28.8 degrees two-theta±0.1 degrees two-theta.

Alternatively, the crystalline form Va of tafamidis may be characterized by an X-ray powder diffraction pattern having peaks at 5.8, 9.5, 13.3, 13.7, 16.3, 19.7, 20.0, and 28.8 degrees two-theta±0.1 degrees two-theta, and further characterized by an X-ray powder diffraction pattern having one, two, three, four, five, or six additional peaks selected from: 23.3, 23.4, 23.7, 25.1, and 26.6 degrees two-theta±0.1 degrees two-theta. In embodiments, crystalline Form Va of tafamidis may be characterized by an X-ray powder diffraction pattern having peaks at 5.8, 9.5, 13.3, 13.7, 16.3, 19.7, 20.0, 23.3, 23.4, 23.7, 25.1, and 26.6 degrees two-theta±0.1 degrees two-theta. Crystalline form of tafamidis, may be alternatively characterized by an X-ray powder diffraction pattern having peaks at 13.3, 16.3 and 19.7 degrees two-theta. The crystalline form of tafamidis may be further characterized by an X-ray powder diffraction pattern having any one, two, three, four or five additional peaks selected from: 5.8, 9.5, 13.7, 20.0, and 28.8 degrees two-theta. In embodiments the crystalline form of tafamidis may be characterized by an X-ray powder diffraction pattern having peaks at 5.8, 9.5, 13.3, 13.7, 16.3, 19.7, 20.0, and 28.8 degrees two-theta.

Alternatively, the crystalline form Va of tafamidis may be characterized by an X-ray powder diffraction pattern having peaks at 5.8, 9.5, 13.3, 13.7, 16.3, 19.7, 20.0, and 28.8 degrees two-theta, and further characterized by an X-ray powder diffraction pattern having one, two, three, four, five, or six additional peaks selected from: 23.3, 23.4, 23.7, 25.1, and 26.6 degrees two-theta. In embodiments, crystalline Form Va of tafamidis may be characterized by an X-ray powder diffraction pattern having peaks at 5.8, 9.5, 13.3, 13.7, 16.3, 19.7, 20.0, 23.3, 23.4, 23.7, 25.1, and 26.6 degrees two-theta.

In any aspect or embodiment the most intense peak of crystalline form Va is at either 13.7 (optionally ±0.2 degrees 2-theta or 0.1 degrees 2-theta), or at 20.0 (optionally ±0.2 degrees 2-theta or 0.1 degrees 2-theta) degrees 2-theta. In any aspect or embodiment, the peak at 5.8 (optionally ±0.2 degrees 2-theta or 0.1 degrees 2-theta), when present, has an intensity relative to the most intense peak at either 13.7 (optionally ±0.2 degrees 2-theta or 0.1 degrees 2-theta) or 20.0 (optionally ±0.2 degrees 2-theta or 0.1 degrees 2-theta), of 15-60%. In any aspect or embodiment, the peak at 9.5 (optionally ±0.2 degrees 2-theta or 0.1 degrees 2-theta), when present, has an intensity relative to the most intense peak at either 13.7 (optionally ±0.2 degrees 2-theta or 0.1 degrees 2-theta) or 20.0 (optionally ±0.2 degrees 2-theta or 0.1 degrees 2-theta), of 2-10%. In any aspect or embodiment, the peak at 13.3 (optionally ±0.2 degrees 2-theta or 0.1 degrees 2-theta), when present, has an intensity relative to the most intense peak at either 13.7 (optionally ±0.2 degrees 2-theta or 0.1 degrees 2-theta or 20.0 (optionally ±0.2 degrees 2-theta or 0.1 degrees 2-theta, of 4-20%. In any aspect or embodiment, when the peak at 20.0 (optionally ±0.2 degrees 2-theta or 0.1 degrees 2-theta) is the most intense peak (100%), the peak at 13.7 (optionally ±0.2 degrees 2-theta or 0.1 degrees 2-theta), has an intensity relative to the most intense peak, of from 50% up to, but not including, 100%. In any aspect or embodiment, the peak at 16.3 (optionally ±0.2 degrees 2-theta or 0.1 degrees 2-theta), has an intensity relative to the most intense peak at either 13.7 (optionally ±0.2 degrees 2-theta or 0.1 degrees 2-theta) or 20.0 (optionally ±0.2 degrees 2-theta or 0.1 degrees 2-theta), of 10-40%. In any aspect or embodiment, the peak at 19.7 (optionally ±0.2 degrees 2-theta or 0.1 degrees 2-theta), has an intensity relative to the most intense peak at either 13.7 (optionally ±0.2 degrees 2-theta or 0.1 degrees 2-theta) or 20.0 (optionally ±0.2 degrees 2-theta or 0.1 degrees 2-theta), of 10-40%. In any aspect or embodiment, when the peak at 13.7 (optionally ±0.2 degrees 2-theta or 0.1 degrees 2-theta) is the most intense peak (100%), the peak at 20.0 (optionally ±0.2 degrees 2-theta or 0.1 degrees 2-theta), has an intensity relative to the most intense peak of from 60% up to, but not including, 100%. In any aspect or embodiment, the peak at 23.3 (optionally ±0.2 degrees 2-theta or 0.1 degrees 2-theta), has an intensity relative to the most intense peak at either 13.7 (optionally ±0.2 degrees 2-theta or 0.1 degrees 2-theta) or 20.0 (optionally ±0.2 degrees 2-theta or 0.1 degrees 2-theta), of 4-20%. In any aspect or embodiment, the peak at 23.4 (optionally ±0.2 degrees 2-theta or 0.1 degrees 2-theta), has an intensity relative to the most intense peak at either 13.7 (optionally ±0.2 degrees 2-theta or 0.1 degrees 2-theta) or 20.0 (optionally ±0.2 degrees 2-theta or 0.1 degrees 2-theta), of 4-30%. In any aspect or embodiment, the peak at 23.7 (optionally ±0.2 degrees 2-theta or 0.1 degrees 2-theta), has an intensity relative to the most intense peak at either 13.7 (optionally ±0.2 degrees 2-theta or 0.1 degrees 2-theta) or 20.0 (optionally ±0.2 degrees 2-theta or 0.1 degrees 2-theta), of 4-25%. In any aspect or embodiment, the peak at 25.1 (optionally ±0.2 degrees 2-theta or 0.1 degrees 2-theta), has an intensity relative to the most intense peak at either 13.7 (optionally ±0.2 degrees 2-theta or 0.1 degrees 2-theta) or 20.0 (optionally ±0.2 degrees 2-theta or 0.1 degrees 2-theta), of 4-20%. In any aspect or embodiment, the peak at 26.6 (optionally ±0.2 degrees 2-theta or 0.1 degrees 2-theta), has an intensity relative to the most intense peak at either 13.7 (optionally ±0.2 degrees 2-theta or 0.1 degrees 2-theta) or 20.0 (optionally ±0.2 degrees 2-theta or 0.1 degrees 2-theta), of 4-20%. In any aspect or embodiment, the peak at 28.8 (optionally ±0.2 degrees 2-theta or 0.1 degrees 2-theta), has an intensity relative to the most intense peak at either 13.7 (optionally ±0.2 degrees 2-theta or 0.1 degrees 2-theta) or 20.0 (optionally ±0.2 degrees 2-theta or 0.1 degrees 2-theta), of 2-10%.

In any aspect or embodiment, the peak at 5.8 (optionally ±0.2 degrees 2-theta or 0.1 degrees 2-theta), when present, has an intensity relative to the most intense peak at 20.0 (optionally ±0.2 degrees 2-theta or 0.1 degrees 2-theta), of 39±10%. In any aspect or embodiment, the peak at 9.5 (optionally ±0.2 degrees 2-theta or 0.1 degrees 2-theta), when present, has an intensity relative to the most intense peak at 20.0 (optionally ±0.2 degrees 2-theta or 0.1 degrees 2-theta), of 4±10%. In any aspect or embodiment, the peak at 13.3 (optionally ±0.2 degrees 2-theta or 0.1 degrees 2-theta), when present, has an intensity relative to the most intense peak at 20.0 (optionally ±0.2 degrees 2-theta or 0.1 degrees 2-theta, of 9±10%. In any aspect or embodiment, the peak at 13.7 (optionally ±0.2 degrees 2-theta or 0.1 degrees 2-theta), has an intensity relative to the most intense peak at 20.0 (optionally ±0.2 degrees 2-theta or 0.1 degrees 2-theta), of 82±10%. In any aspect or embodiment, the peak at 16.3 (optionally ±0.2 degrees 2-theta or 0.1 degrees 2-theta), has an intensity relative to the most intense peak at 20.0 (optionally ±0.2 degrees 2-theta or 0.1 degrees 2-theta), of 23 f 10%. In any aspect or embodiment, the peak at 19.7 (optionally ±0.2 degrees 2-theta or 0.1 degrees 2-theta), has an intensity relative to the most intense peak at either 13.7 (optionally ±0.2 degrees 2-theta or 0.1 degrees 2-theta) or 20.0 (optionally ±0.2 degrees 2-theta or 0.1 degrees 2-theta), of 25±10%. In any aspect or embodiment, the peak at 23.3 (optionally ±0.2 degrees 2-theta or 0.1 degrees 2-theta), has an intensity relative to the most intense peak at 20.0 (optionally ±0.2 degrees 2-theta or 0.1 degrees 2-theta), of 11±10%. In any aspect or embodiment, the peak at 23.4 (optionally ±0.2 degrees 2-theta or 0.1 degrees 2-theta), has an intensity relative to the most intense peak at 20.0 (optionally ±0.2 degrees 2-theta or 0.1 degrees 2-theta), of 12±10%. In any aspect or embodiment, the peak at 23.7 (optionally ±0.2 degrees 2-theta or 0.1 degrees 2-theta), has an intensity relative to the most intense peak at 20.0 (optionally ±0.2 degrees 2-theta or 0.1 degrees 2-theta), of 13±10%. In any aspect or embodiment, the peak at 25.1 (optionally ±0.2 degrees 2-theta or 0.1 degrees 2-theta), has an intensity relative to the most intense peak at 20.0 (optionally ±0.2 degrees 2-theta or 0.1 degrees 2-theta), of 10±10%. In any aspect or embodiment, the peak at 26.6 (optionally ±0.2 degrees 2-theta or 0.1 degrees 2-theta), has an intensity relative to the most intense peak at 20.0 (optionally ±0.2 degrees 2-theta or 0.1 degrees 2-theta), of 9±10%. In any aspect or embodiment, the peak at 28.8 (optionally ±0.2 degrees 2-theta or 0.1 degrees 2-theta), has an intensity relative to the most intense peak at 20.0 (optionally ±0.2 degrees 2-theta or 0.1 degrees 2-theta), of 4±10%.

In any aspect or embodiment, the peak at 5.8 (optionally ±0.2 degrees 2-theta or 0.1 degrees 2-theta), when present, has an intensity relative to the most intense peak at 20.0 (optionally ±0.2 degrees 2-theta or 0.1 degrees 2-theta), of 39±5%. In any aspect or embodiment, the peak at 9.5 (optionally ±0.2 degrees 2-theta or 0.1 degrees 2-theta), when present, has an intensity relative to the most intense peak at 20.0 (optionally ±0.2 degrees 2-theta or 0.1 degrees 2-theta), of 4±5%. In any aspect or embodiment, the peak at 13.3 (optionally ±0.2 degrees 2-theta or 0.1 degrees 2-theta), when present, has an intensity relative to the most intense peak at 20.0 (optionally ±0.2 degrees 2-theta or 0.1 degrees 2-theta, of 9±5%. In any aspect or embodiment, the peak at 13.7 (optionally ±0.2 degrees 2-theta or 0.1 degrees 2-theta), has an intensity relative to the most intense peak at 20.0 (optionally ±0.2 degrees 2-theta or 0.1 degrees 2-theta), of 82±5%. In any aspect or embodiment, the peak at 16.3 (optionally ±0.2 degrees 2-theta or 0.1 degrees 2-theta), has an intensity relative to the most intense peak at 20.0 (optionally ±0.2 degrees 2-theta or 0.1 degrees 2-theta), of 23±5%. In any aspect or embodiment, the peak at 19.7 (optionally ±0.2 degrees 2-theta or 0.1 degrees 2-theta), has an intensity relative to the most intense peak at either 13.7 (optionally ±0.2 degrees 2-theta or 0.1 degrees 2-theta) or 20.0 (optionally ±0.2 degrees 2-theta or 0.1 degrees 2-theta), of 25±5%. In any aspect or embodiment, the peak at 23.3 (optionally ±0.2 degrees 2-theta or 0.1 degrees 2-theta), has an intensity relative to the most intense peak at 20.0 (optionally ±0.2 degrees 2-theta or 0.1 degrees 2-theta), of 11±5%. In any aspect or embodiment, the peak at 23.4 (optionally ±0.2 degrees 2-theta or 0.1 degrees 2-theta), has an intensity relative to the most intense peak at 20.0 (optionally ±0.2 degrees 2-theta or 0.1 degrees 2-theta), of 12±5%. In any aspect or embodiment, the peak at 23.7 (optionally ±0.2 degrees 2-theta or 0.1 degrees 2-theta), has an intensity relative to the most intense peak at 20.0 (optionally ±0.2 degrees 2-theta or 0.1 degrees 2-theta), of 13±5%. In any aspect or embodiment, the peak at 25.1 (optionally ±0.2 degrees 2-theta or 0.1 degrees 2-theta), has an intensity relative to the most intense peak at 20.0 (optionally ±0.2 degrees 2-theta or 0.1 degrees 2-theta), of 10±5%. In any aspect or embodiment, the peak at 26.6 (optionally ±0.2 degrees 2-theta or 0.1 degrees 2-theta), has an intensity relative to the most intense peak at 20.0 (optionally ±0.2 degrees 2-theta or 0.1 degrees 2-theta), of 9±5%. In any aspect or embodiment, the peak at 28.8 (optionally ±0.2 degrees 2-theta or 0.1 degrees 2-theta), has an intensity relative to the most intense peak at 20.0 (optionally ±0.2 degrees 2-theta or 0.1 degrees 2-theta), of 4±5%.

In any aspect or embodiment, the peak at 5.8 (optionally ±0.2 degrees 2-theta or 0.1 degrees 2-theta), when present, has an intensity relative to the most intense peak at 20.0 (optionally ±0.2 degrees 2-theta or 0.1 degrees 2-theta), of 39%. In any aspect or embodiment, the peak at 9.5 (optionally ±0.2 degrees 2-theta or 0.1 degrees 2-theta), when present, has an intensity relative to the most intense peak at 20.0 (optionally ±0.2 degrees 2-theta or 0.1 degrees 2-theta), of 4%. In any aspect or embodiment, the peak at 13.3 (optionally f 0.2 degrees 2-theta or 0.1 degrees 2-theta), when present, has an intensity relative to the most intense peak at 20.0 (optionally ±0.2 degrees 2-theta or 0.1 degrees 2-theta, of 9%. In any aspect or embodiment, the peak at 13.7 (optionally ±0.2 degrees 2-theta or 0.1 degrees 2-theta), has an intensity relative to the most intense peak at 20.0 (optionally ±0.2 degrees 2-theta or 0.1 degrees 2-theta), of 82%. In any aspect or embodiment, the peak at 16.3 (optionally f 0.2 degrees 2-theta or 0.1 degrees 2-theta), has an intensity relative to the most intense peak at 20.0 (optionally ±0.2 degrees 2-theta or 0.1 degrees 2-theta), of 23%. In any aspect or embodiment, the peak at 19.7 (optionally ±0.2 degrees 2-theta or 0.1 degrees 2-theta), has an intensity relative to the most intense peak at either 13.7 (optionally ±0.2 degrees 2-theta or 0.1 degrees 2-theta) or 20.0 (optionally ±0.2 degrees 2-theta or 0.1 degrees 2-theta), of 25%. In any aspect or embodiment, the peak at 23.3 (optionally ±0.2 degrees 2-theta or 0.1 degrees 2-theta), has an intensity relative to the most intense peak at 20.0 (optionally ±0.2 degrees 2-theta or 0.1 degrees 2-theta), of 115%. In any aspect or embodiment, the peak at 23.4 (optionally f 0.2 degrees 2-theta or 0.1 degrees 2-theta), has an intensity relative to the most intense peak at 20.0 (optionally ±0.2 degrees 2-theta or 0.1 degrees 2-theta), of 12%. In any aspect or embodiment, the peak at 23.7 (optionally ±0.2 degrees 2-theta or 0.1 degrees 2-theta), has an intensity relative to the most intense peak at 20.0 (optionally ±0.2 degrees 2-theta or 0.1 degrees 2-theta), of 13%. In any aspect or embodiment, the peak at 25.1 (optionally ±0.2 degrees 2-theta or 0.1 degrees 2-theta), has an intensity relative to the most intense peak at 20.0 (optionally ±0.2 degrees 2-theta or 0.1 degrees 2-theta), of 10%. In any aspect or embodiment, the peak at 26.6 (optionally ±0.2 degrees 2-theta or 0.1 degrees 2-theta), has an intensity relative to the most intense peak at 20.0 (optionally ±0.2 degrees 2-theta or 0.1 degrees 2-theta), of 9%. In any aspect or embodiment, the peak at 28.8 (optionally ±0.2 degrees 2-theta or 0.1 degrees 2-theta), has an intensity relative to the most intense peak at 20.0 (optionally ±0.2 degrees 2-theta or 0.1 degrees 2-theta), of 4%.

In any aspect or embodiment, crystalline tafamidis form Va is characterized by an XRPD pattern having the following 2-theta values and relative intensities:

| 2-theta value (degrees 2-theta) | |
|---|---|
| (optionally ±0.2 degrees 2- | Relative Intensity (%) |
| theta or ±0.1 degrees 2-theta | (optionally ±10%, or ±5%) |
| 5.8 | 39 |
| 9.5 | 4 |
| 13.3 | 9 |
| 13.7 | 82 |
| 16.3 | 23 |
| 19.7 | 25 |
| 20.0 | 100 |
| 23.3 | 11 |
| 23.4 | 12 |
| 23.7 | 13 |
| 25.1 | 10 |
| 26.6 | 9 |
| 28.8 | 4 |

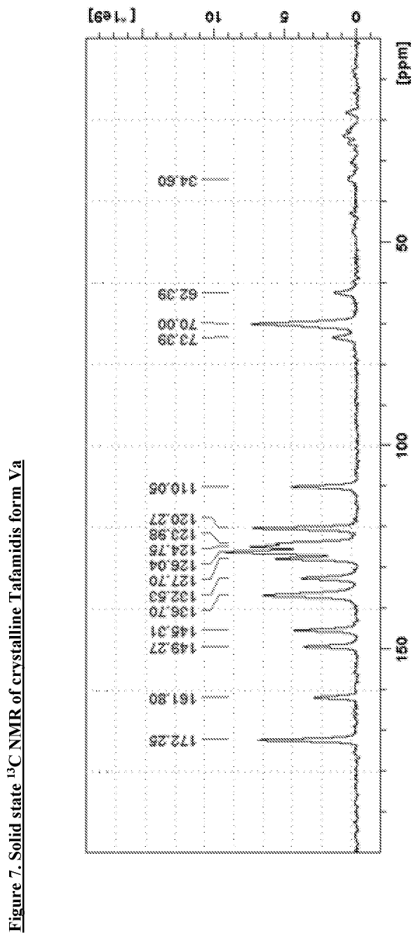

Crystalline form Va of tafamidis may alternatively, or additionally with any of the above embodiments, be characterized by a solid state ¹³C NMR spectrum having peaks at 161.8, 145.3, 132.5, 126.0, 120.3±0.2 ppm, and/or a solid state ¹³C NMR spectrum having chemical shift differences between a reference peak at 70.0±0.2 ppm of: 91.8, 75.3, 62.5, 56.0, 50.3±0.1 ppm respectively. Crystalline form Va of tafamidis may alternatively, or additionally with any of the above embodiments, be characterized by a solid state ¹³C NMR spectrum having peaks at 34.6, 62.4, 70.0, 73.4, 110.1, 120.3, 124.0, 124.8, 126.0, 127.7, 132.56, 136.7, 145.3, 149.3, 161.8, and 172.3±0.2 ppm, or a solid state ¹³C NMR spectrum substantially as depicted in FIG. 7.

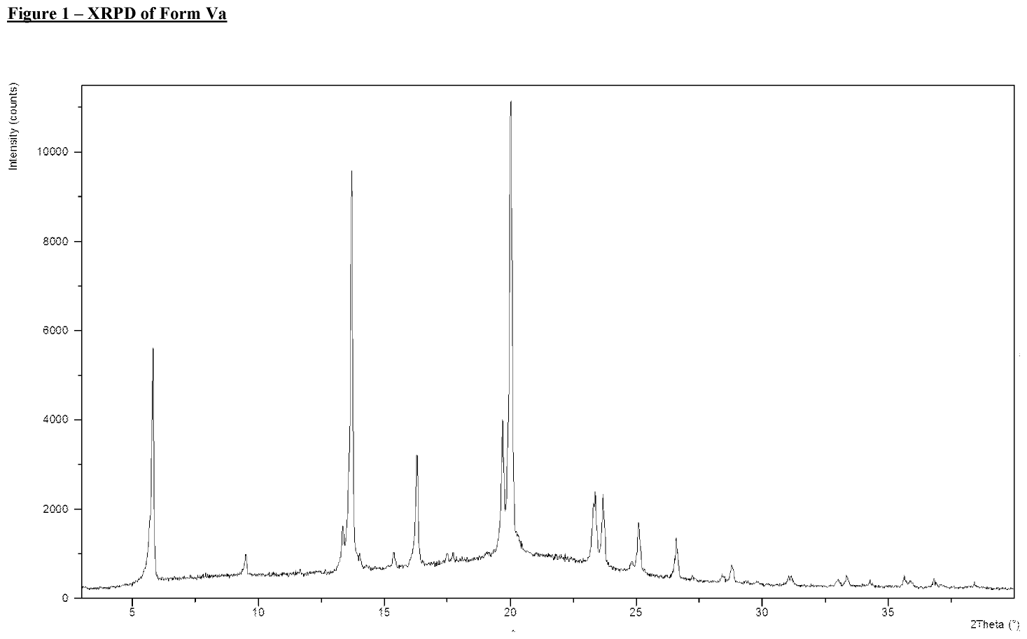

In any aspect or embodiment, crystalline tafamidis form Va is characterized by an XRPD pattern substantially as depicted in FIG. 1.

In any aspect or embodiment, crystalline tafamidis form Va may be a hydrate.

In any aspect or embodiment the crystalline tafamidis form Va may contain about 1% to about 8% (w/w) water, about 2% to about 7% (w/w) water, about 2% to about 7.5% (w/w) water, about 3% to about 7% (w/w) water, about 5% to about 6.5% (w/w) water, about 5.5% to about 6% (w/w) water, about 1% to about 6% (w/w), about 2% to about 6% (w/w), about 3% to about 6% (w/w), about 4% to about 6% (w/w), about 5% to about 6% (w/w), about 5.5% to about 6% (w/w), or about 6% (w/w), water.

In any aspect or embodiment the crystalline tafamidis form Va may be substantially free of any other crystalline forms of tafamidis.

In any aspect or embodiment the crystalline tafamidis form Va may contain: about 20% (w/w) or less, about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of any other crystalline form of tafamidis, preferably as measured by XRPD.

In any aspect or embodiment the crystalline tafamidis form Va may be substantially free of any amorphous forms of tafamidis.

In any aspect or embodiment the crystalline tafamidis form Va may contain: about 20% (w/w) or less, about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of any amorphous form of tafamidis, preferably as measured by XRPD.

In any aspect or embodiment the crystalline tafamidis form Va may contain: about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of any solvated organic solvent in the crystal structure.

A further aspect of the disclosure comprises a process for preparing a crystalline form of tafamidis as defined in any aspect or embodiment, which comprises crystallizing tafamidis from a mixture comprising a polyethylene glycol (PEG). In particular, the process may comprise (a) providing a mixture comprising tafamidis in a solvent comprising polyethylene glycol; (b) stirring the mixture; and (c) optionally isolating the solid. The mixture may be a slurry. The starting material is preferably tafamidis Form V, most preferably anhydrous tafamidis Form V. The starting material for the process according to any aspect or embodiment, may be characterized by a PXRD pattern having peaks at 6.0, 19.9, 20.6, 23.9 and 29.2 degrees 2-theta±0.2 degrees 2-theta, or by a PXRD pattern having peaks at 6.0, 19.9, 20.6, 23.9 and 29.2 degrees 2-theta±0.2 degrees 2-theta, and also having one, two, three or four additional peaks at 17.8, 25.8, 27.3 and 31.1 degrees 2-theta±0.2 degrees 2-theta; or alternatively a PXRD pattern having peaks at 6.0, 17.8, 19.9, 20.6, 23.9, 25.8, 27.3, 29.2 and 31.1 degrees 2-theta±0.2 degrees 2-theta.

In any aspect or embodiment of the process, the starting material may be alternatively or additionally characterized by a solid state ¹³C NMR spectrum having peaks at 171.5, 161.0, 149.1, 144.7, 131.0±0.2 ppm and/or a solid state ¹³C NMR spectrum having the following chemical shift absolute differences from a reference peak at 109.5 ppm±0.2 ppm of 62.1, 51.6, 39.6, 35.2, 21.5±0.1 ppm or a solid state ¹³C NMR spectrum substantially as depicted in FIG. 4.

According to any aspect or embodiment of the process of the present invention, the polyethylene glycol may be PEG-400 or PEG-300, or PEG-200. The solvent may further comprise water. The volume ratio of polyethylene glycol to water may be from: about 70:30 to about 30:70, about 80:20 to about 20:80, about 90:10 to about 99:1, about 90:10 to about 99.8:0.2, about 90:10 to about 99.5:0.5, about 92:8 to about 99:1, about 92:10 to about 98:2, about 94:6 to about 98:2, or about 95:5.

According to any aspect or embodiment of the process of the present invention, the mixture may be stirred at a temperature of: about 10° C. to about 45° C., about 15° C. to about 40° C., about 18° C. to about 30° C., or about 20° C. to about 25° C. Optionally, the mixture can be stirred for a period of: about 2 hours to about 72 hours, about 5 hours to about 8 to about 36 hours, about 12 to about 30 hours, or about 14 to about 28 hours

According to any aspect or embodiment of the process of the present invention, tafamidis Form Va may be isolated, preferably by filtration, centrifugation, or decantation, preferably by filtration.

According to any aspect or embodiment, the process may further comprise combining the product with at least one pharmaceutically acceptable excipient to provide a pharmaceutical formulation, preferably wherein the formulation is a tablet or capsule.

The disclosure further encompasses the use of crystalline Form V of tafamidis as described above, as a starting material for preparing a crystalline hydrate of tafamidis.

The disclosure further encompasses the use of crystalline Form V of tafamidis as defined above, as a starting material for preparing crystalline Form Va of tafamidis. The crystalline form Va of tafamidis may be defined according to any aspect or embodiment of the disclosure.

The disclosure encompasses a product obtainable by any of the herein disclosed processes.

The disclosure further encompasses a pharmaceutical composition comprising crystalline form Va as defined in any aspect or embodiment herein, and at least one pharmaceutically acceptable excipient. The at least one pharmaceutically acceptable excipient may be selected from one or more of: a solubilizer, a surfactant, and an emulsifier.

The disclosure further encompasses a pharmaceutical composition comprising crystalline form Va as defined in any aspect or embodiment herein and a pharmaceutical excipient, wherein the excipient comprises a polyethylene glycol, preferably PEG-400, or PEG-300, or PEG-200, and more preferably PEG-400.

Optionally, the pharmaceutical composition may comprise at least one non-ionic surfactant, preferably a hydrophilic non-ionic surfactant, particularly an ethoxylated sorbitan ester, more particularly a polyoxyethylene sorbitan ester, and preferably a polysorbate selected from polysorbate 20, polysorbate 60, and polysorbate 80, most preferably polysorbate 20 or polysorbate 80. Optionally, the pharmaceutical composition may comprise a non-ionic emulsifier, preferably a lipophilic non-ionic emulsifier, particularly a sorbitan fatty acid ester, and more preferably sorbitan monostearate or sorbitan monooleate, preferably sorbitan monooleate. Optionally, the pharmaceutical composition may comprise a polyvinylpyrrolidone, preferably a polyvinylpyrrolidone having a molecular weight of between about 45 kDa to about 3000 kDa, about 60 kDa to about 2000 kDa, about 80 kDa to about 180 kDa, or about 100 kDa to about 150 kDa.

The present disclosure further encompasses the use of a crystalline product as described in any aspect or embodiment of the present disclosure, for the preparation of a pharmaceutical composition and/or formulation, preferably wherein the pharmaceutical formulation is a tablet or capsule, more preferably a capsule.

The pharmaceutical compositions of the present disclosure may be prepared by combining a crystalline product according to any aspect or embodiment of the present disclosure, with at least one pharmaceutically acceptable excipient, wherein the pharmaceutically acceptable excipient may be as described in any aspect or embodiment disclosed herein.

The present disclosure encompasses a crystalline product as described in any embodiment or aspect of the present disclosure, or a pharmaceutical composition as described in any aspect or embodiment of the present disclosure, for use as a medicament.

The present disclosure encompasses a crystalline product as described in any embodiment or aspect of the present disclosure, or a pharmaceutical composition as described in any aspect or embodiment of the present disclosure, for use in the treatment of transthyretin-mediated amyloidosis, preferably for use in the treatment of familial amyloid polyneuropathy, cardiomyopathy or wild type or hereditary transthyretin-mediated amyloidosis, or transthyretin amyloidosis in adult patients with stage 1 symptomatic polyneuropathy.

Also provided is a method of treating transthyretin-mediated amyloidosis, preferably for use in the treatment of familial amyloid polyneuropathy, cardiomyopathy or wild type or hereditary transthyretin-mediated amyloidosis, or transthyretin amyloidosis in adult patients with stage 1 symptomatic polyneuropathy, comprising administering a therapeutically effective amount of a crystalline product as described in any aspect or embodiment disclosed herein, or a pharmaceutical composition as described in any aspect or embodiment disclosed herein, to a subject in need of the treatment.

Further provided is the use of a crystalline product as described in any aspect or embodiment herein, in the preparation of another solid state form of tafamidis, tafamidis solvate, or tafamidis salt.

The disclosure further provides a process for preparing a solid state form of tafamidis, tafamidis solvate, or tafamidis salt, comprising preparing a crystalline product according to any aspect or embodiment or embodiment of the present disclosure, and converting it to another a solid state form thereof.

In any aspect or embodiment of the present disclosure, the crystalline Form Va of tafamidis described herein may be polymorphically pure or may be substantially free of any other solid state forms of tafamidis. In any aspect or embodiment of the present disclosure, the crystalline Form Va of tafamidis, may contain: about 20% (w/w) or less, about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of any other solid state form of tafamidis, preferably as measured by XRPD. Thus, the disclosed crystalline Form Va of tafamidis described herein may be substantially free of any other solid state forms of tafamidis, and may contain greater than about 80% (w/w), greater than about 90% (w/w), greater than about 95% (w/w), greater than about 98% (w/w), greater than about 99% (w/w), or about 100% of tafamidis Form Va.

The present disclosure also provides the use of the crystalline form of tafamidis according to any of the disclosed embodiments or aspects for preparing other solid state forms of tafamidis, tafamidis salts and solid state forms thereof.

The present disclosure further encompasses processes for preparing other solid state forms of tafamidis, or solid state forms thereof, as well as other tafamidis salts or solid state forms thereof. The process includes preparing the solid state form of the present disclosure, and converting it to other solid state forms of tafamidis. Alternatively, the process includes preparing the solid state form of the present disclosure, and converting it to tafamidis salt. The conversion can be done, for example, by a process including reacting the obtained tafamidis with an appropriate base such as meglumine, alkali/alkaline earth metal bases such as potassium, sodium, calcium, magnesium, ammonia or alkyl amines (in embodiments C₁₋₆ mono-, di- or trialkylamines). In embodiments, the alkali/alkaline earth metal bases are selected from the group consisting of potassium hydroxide, potassium carbonate, potassium hydrogen carbonate, sodium hydroxide, sodium carbonate, sodium hydrogen carbonate, calcium hydroxide or magnesium hydroxide.

In another embodiment the present disclosure encompasses the above described solid state form of tafamidis, for use in the preparation of pharmaceutical compositions and/or formulations, in embodiments for the treatment of transthyretin-mediated amyloidosis. In embodiments the present disclosure encompasses the use of the above described solid state form of tafamidis for the preparation of a pharmaceutical composition comprising tafamidis or salt thereof.

In another embodiment the present disclosure encompasses the use of the above described solid state form of tafamidis, for the preparation of pharmaceutical compositions and/or formulations, in embodiments oral formulations, e.g., tablets or capsules. In embodiments the present disclosure encompasses the above described solid state form of tafamidis, for the preparation of a pharmaceutical composition or formulation, in embodiments an oral formulation in the form of a dispersion including tafamidis or salt thereof.

The present disclosure further provides pharmaceutical compositions including the solid state form of tafamidis, according to the present disclosure.

In yet another embodiment, the present disclosure encompasses pharmaceutical formulations comprising the solid state forms of tafamidis as described herein, and at least one pharmaceutically acceptable excipient.

In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present disclosure, the active ingredient and any other solid excipients are dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present disclosure include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present disclosure can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, in embodiments a powdered or granulated solid composition of the disclosure, within either a hard or soft shell. The shell can be made from gelatin and optionally contain a plasticizer such as glycerin and sorbitol, and an opacifying agent or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, in embodiments water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of tafamidis may be formulated for administration to a mammal, in embodiments a human. Tafamidis can be formulated, for example, as a viscous liquid solution or suspension, in embodiments a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others, including those disclosed in Ansel et al., Pharmaceutical Dosage Forms and Drug Delivery Systems, 7th ed. The present disclosure encompasses a process to prepare said formulations of tafamidis by combining at least one of the above solid state forms and at least one pharmaceutically acceptable excipient.

The solid state form as defined herein, as well as the pharmaceutical compositions or formulations of tafamidis can be used as medicaments, in embodiments for the treatment of transthyretin-mediated amyloidosis.

The present disclosure also provides a method of treating transthyretin-mediated amyloidosis, by administering a therapeutically effective amount of the solid state form of tafamidis of the present disclosure, or at least one of the above pharmaceutical compositions or formulations, to a subject suffering from transthyretin-mediated amyloidosis, in embodiments cardiomyopathy of wild type or hereditary transthyretin-mediated amyloidosis, or transthyretin amyloidosis in adult patients with stage 1 symptomatic polyneuropathy, to delay peripheral neurologic impairment or otherwise in need of the treatment.

The present disclosure also provides the use of the solid state form of tafamidis of the present disclosure, or at least one of the above pharmaceutical compositions or formulations for the manufacture of a medicament for treating transthyretin-mediated amyloidosis, in embodiments cardiomyopathy of wild type or hereditary transthyretin-mediated amyloidosis, or transthyretin amyloidosis in adult patients with stage 1 symptomatic polyneuropathy, to delay peripheral neurologic impairment.

Having described the invention with reference to certain preferred embodiments, other embodiments will become apparent to one skilled in the art from consideration of the specification. The invention is further illustrated by reference to the following examples describing in detail the preparation of the composition and methods of use of the composition. It will be apparent to those skilled in the art that many modifications, both to materials and methods, may be practiced without departing from the scope of the disclosure.

### Analytical Methods

### Powder X-Ray Diffraction Pattern (“PXRD”) Method:

Sample after being powdered in a mortar and pestle is applied directly on a silicon plate holder. The X-ray powder diffraction pattern was measured with Philips X'Pert PRO X-ray powder diffractometer, equipped with Cu irradiation source=1.541874 Å. (\acute{Å}ngström), X'Celerator (2.022° 2θ) detector. Scanning parameters: angle range: 3-40 deg., step size 0.0167, time per step 37 s, continuous scan. Measurement temperature was 25° C.±3° C.

¹³C Solid State Nuclear Magnetic Resonance (“ss-NMR” or ¹³C Solid State NMR) Method:

13C CP/MAS and 1H MAS NMR spectra were recorded at 16.4 T using a Bruker Avance NEO 700 SB NMR spectrometer (Karlsruhe, Germany, 2021) with 3.2 mm probehead. The 13C CP/MAS NMR spectra employing cross-polarization were acquired using the standard cross-polarization pulse scheme at spinning frequency of 18 kHz. The 13C scale was referenced to glycine (176.03 ppm for 13C).

### EXAMPLES

Tafamidis can be prepared according to the procedure described in U.S. Pat. No. 7,214,695. Tafamidis Form V can be prepared according to any of the procedures described in International Publication No. WO2020/232325.

### Example 1: Preparation of Tafamidis Form Va

1 gram of Tafamidis Form V (anhydrous) was placed into a glass beaker. 10 ml of PEG-400 was added. Obtained slurry was mixed at room temperature using a magnetic stirrer for 24 hours and analysed by XRPD. XRPD pattern is given in FIG. 1.

### Example 2: Preparation of Tafamidis Form Va

100 mg of Tafamidis Form V (anhydrous) was placed into a glass beaker. 1 ml of PEG-400/water mixture (95:5 volume ratio) was added. Obtained slurry was mixed at room temperature using a magnetic stirrer for 16 hours and analysed by XRPD and corresponds to Form Va.

### Example 3: Preparation of Tafamidis Form V

Tafamidis (1.0 gram, 3.25 mmol) was dissolved in mixture of solvents toluene/N-methyl-2-pyrrolidone (NMP) (15 V; 15% NMP) by heating up to 70-75° C. The solution was spontaneously cooled down to 30° C. and slowly added to cold methanol (30 V) at 0-5° C. Crystallization was momentary. The obtained suspension was stirred at 0-5° C. for 2 hours. The obtained crystals were isolated by vacuum filtration. Obtained solid was washed with methanol (10 V) and dried in vacuum dryer at 80° C., 20 mbar for 6 hours (0.9 grams of crystals was obtained, chrom. purity 99%). Obtained solid was analyzed by XRPD. Tafamidis form V (anhydrous) was obtained.

### Example 4: Preparation of Tafamidis Form V

Tafamidis (498 mg) was dissolved in THF (28 ml) at room temperature. Antisolvent (Methanol) was cooled to 0° C. (ice bath) and added dropwise into solution (92 ml). Crystallization was momentary. Suspension was stirred for one hour and then isolated by vacuum filtration (427 mg). Obtained solid was washed with solvent mixture THF:Methanol (1:4). Obtained solid was analyzed by XRD. Tafamidis form V (Methanol solvate) was obtained. Obtained solid was subjected to heating in vacuum dryer at 80° C. for 2 hours. Obtained solid was analyzed by XRPD. Tafamidis Form V (anhydrous) was obtained.

### Example 5: Preparation of Tafamidis Form V

4-(3,5-dichlorobenzamido)-3-hydroxybenzoic acid (25.00 grams; 76.7 mmol), toluene (281.3 ml), N-methyl-pyrrolidone (93.8 ml) and methanesulfonic acid (14.9 ml; 230.0 mmol: 3.0 eq) were charged into reactor at 20-25° C. The reaction mixture was heated to reflux temperature (117-119° C.) and stirred until the reaction was completed (about 15 hours). The reaction mixture was then cooled down to 100-110° C. and pH was adjusted to 1.4-1.8 with triethylamine addition (32 ml; 230.0 mmol; 3.0 eq.). The reaction mixture was added dropwise to cold methanol (750 ml; previously cooled down to 0-5° C.) during 1 hour. Crystallization occurred. The suspension was stirred at 0-5° C. for additional 3 hours. Crystals of Tafamidis methanol solvate were filtered off over Buchner funnel and washed with methanol (2×100 ml). Wet crystals were suspended in fresh methanol (375 ml) at 20-25° C. for 3-6 hours. Crystals of Tafamidis methanol solvate were filtered off over Buchner funnel, washed with methanol (2×50 ml) and dried at 80° C., 20 mbar, 10 hours. White crystals of Tafamidis form V (anhydrous) were obtained.

### Example 6: Preparation of Tafamidis Form Va

Tafamidis Form V (1 gram) was slurried in PEG-400 (10 ml) at 25° C. for 24 hours, and after 7 days. The resulting solids were each confirmed by XRPD to be Tafamidis Form Va. The suspension was centrifuged and the solid was filtered under vacuum, and washed with portions of methanol/water (90/10 v/v ratio). The resulting solids were each confirmed by XRPD to be Tafamidis Form Va.

### Example 7: Stability of Form Va

50 mg of samples of Form Va and Form 4 were placed into glass vials. 1 ml of PEG 400 was added at room temperature. The slurries were each mixed using a magnetic stirrer. The resulting samples were analysed by XRPD after 2 hours and after 7 days. As shown in FIG. 5, Form 4 rapidly converts to Form 1 within 2 hours, whereas Form Va is stable for at least 7 days.

1. A crystalline form of tafamidis, designated form Va, which is characterized by at least one of:
(i) an X-ray powder diffraction pattern having peaks at 13.3, 16.3 and 19.7 degrees two-theta±0.2 degrees two-theta;
(ii) a solid state ¹³C NMR spectrum having characteristic peaks at 161.8, 145.3, 132.5, 126.0, 120.3±0.2 ppm; or
(iii) a solid state ¹³C NMR spectrum having chemical shift differences between a reference peak at 70.0±0.2 ppm of: 91.8, 75.3, 62.5, 56.0, 50.3±0.1 ppm, respectively.

2. The crystalline form of tafamidis according to claim 1, which is further characterized by an X-ray powder diffraction pattern having an additional peak at 5.8 degrees two-theta±0.2 degrees two-theta.

3. The crystalline form of tafamidis according to claim 1, which is further characterized by an X-ray powder diffraction pattern having an additional peak at 9.5 degrees two-theta±0.2 degrees two-theta.

4. The crystalline form of tafamidis according to claim 1, which is further characterized by an X-ray powder diffraction pattern having an additional peak at 13.7 degrees two-theta±0.2 degrees two-theta.

5. The crystalline form of tafamidis according to claim 1, which is further characterized by an X-ray powder diffraction pattern having an additional peak at 20.0 degrees two-theta±0.2 degrees two-theta.

6. The crystalline form of tafamidis according to claim 1, which is further characterized by an X-ray powder diffraction pattern having an additional peak at about 28.8 degrees two-theta±0.2 degrees two-theta.

7. The crystalline form of tafamidis according to claim 1, which is characterized by an X-ray powder diffraction pattern having peaks at 5.8, 9.5, 13.3, 13.7, 16.3, 19.7, 20.0, and 28.8 degrees two-theta±0.2 degrees two-theta.

8. The crystalline form of tafamidis according to claim 7, which is further characterized by an X-ray powder diffraction pattern having one, two, three, four, or five additional peaks selected from: 23.3, 23.4, 23.7, 25.1, and 26.6 degrees two-theta±0.2 degrees two-theta.

9. The crystalline form of tafamidis according to claim 8, which is characterized by an X-ray powder diffraction pattern having peaks at 5.8, 9.5, 13.3, 13.7, 16.3, 19.7, 20.0, 23.3, 23.4, 23.7, 25.1, and 26.6 degrees two-theta±0.2 degrees two-theta.

10-27. (canceled)

28. The crystalline form of tafamidis according to claim 1, wherein the specified 2-theta values, when present, have the following intensity ranges relative to the most intense peak, wherein only one of the peaks at 13.7 or 20.0 can have a relative intensity of 100%:

| 2-theta value (degrees 2-theta) | |
|---|---|
| (optionally ±0.2 degrees 2- | Intensity relative to the |
| theta or ±0.1 degrees 2-theta | most intense peak (%) |
| 5.8 | 15-60 |
| 9.5 | 2-10 |
| 13.3 | 4-20 |
| 13.7 | 50-100 |
| 16.3 | 10-40 |
| 19.7 | 10-40 |
| 20.0 | 60-100 |
| 23.3 | 4-20 |
| 23.4 | 4-30 |
| 23.7 | 4-25 |
| 25.1 | 4-20 |
| 26.6 | 4-20 |
| 28.8 | 2-10 |

29. (canceled)

30. (canceled)

31. The crystalline form of tafamidis according to claim 1, which is characterized by an XRPD pattern substantially as depicted in FIG. 1 or a solid state ¹³C NMR spectrum substantially as depicted in FIG. 7.

32. The crystalline form of tafamidis according to claim 1, which is a hydrate.

33. The crystalline form of tafamidis according to claim 32, which contains from: about 1% to about 8% (w/w) water, about 2% to about 7% (w/w) water, about 2% to about 7.5% (w/w) water, about 3% to about 7% (w/w) water, about 5% to about 6.5% (w/w) water, about 5.5% to about 6% (w/w) water, about 1% to about 6% (w/w), about 2% to about 6% (w/w), about 3% to about 6% (w/w), about 4% to about 6% (w/w), about 5% to about 6% (w/w), about 5.5% to about 6% (w/w), or about 6% (w/w) water.

34. The crystalline form of tafamidis according to claim 1, which is substantially free of any other crystalline forms of tafamidis.

35. The crystalline form of tafamidis according to claim 34, which contains: about 20% (w/w) or less, about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of any other crystalline forms of tafamidis.

36. The crystalline form of tafamidis according to claim 1, which is substantially free of any amorphous form of tafamidis, wherein the crystalline form contains: about 20% (w/w) or less, about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of any other amorphous of tafamidis.

37. The crystalline form of tafamidis according to claim 36, which contains: about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of any solvated organic solvent in the crystal structure.

38. A process for preparing a crystalline form of tafamidis as defined in claim_1, which comprises crystallizing tafamidis from a mixture comprising a polyethylene glycol (PEG).

39. The process according to claim 38, wherein the process comprises:
(a) providing a mixture comprising a starting material including tafamidis in a solvent comprising polyethylene glycol;
(b) stirring the mixture; and
(c) optionally isolating the solid.

40. The process according to claim 38, wherein the mixture is a slurry.

41. The process according to claim 38, wherein the starting material is tafamidis Form V.

42. The process according to claim 38, wherein the starting material is anhydrous tafamidis Form V.

43. The process according to claim 38, wherein the starting material is characterized by an X-ray powder diffraction pattern having peaks at 6.0, 19.9, 20.6, 23.9 and 29.2 degrees 2-theta±0.2 degrees 2-theta.

44. The process according to claim 43, wherein the starting material is characterized by an X-ray powder diffraction pattern having peaks at 6.0, 19.9, 20.6, 23.9 and 29.2 degrees 2-theta±0.2 degrees 2-theta, and also having one, two, three or four additional peaks at 17.8, 25.8, 27.3 and 31.1 degrees 2-theta±0.2 degrees 2-theta.

45. The process according to claim 38, wherein the starting material is characterized by an X-ray powder diffraction pattern having peaks at 6.0, 17.8, 19.9, 20.6, 23.9, 25.8, 27.3, 29.2 and 31.1 degrees 2-theta±0.2 degrees 2-theta.

46. The process according to claim 38, wherein the Form V of tafamidis is characterized by a solid state ¹³C NMR spectrum having peaks at 171.5, 161.0, 149.1, 144.7, 131.0±0.2 ppm and/or a solid state ¹³C NMR spectrum having the following chemical shift absolute differences from a reference peak at 109.5 ppm±0.2 ppm of 62.1, 51.6, 39.6, 35.2, 21.5±0.1 ppm or a solid state ¹³C NMR spectrum substantially as depicted in FIG. 4.

47. The process according to claim 38 wherein the polyethylene glycol is PEG-400 or PEG-300, or PEG-200.

48. The process according to claim 39, wherein the solvent further comprises water.

49. The process according to claim 48, wherein the volume ratio of polyethylene glycol to water is from: about 70:30 to about 30:70, about 80:20 to about 20:80, about 90:10 to about 99:1, about 90:10 to about 99.8:0.2, about 90:10 to about 99.5:0.5, about 92:8 to about 99:1, about 92:10 to about 98:2, about 94:6 to about 98:2, or about 95:5.

50. The process according to claim 38, wherein the mixture is stirred at a temperature of: about 10° C. to about 45° C., about 15° C. to about 40° C., about 18° C. to about 30° C., or about 20° C. to about 25° C.

51. The process according to claim 38, wherein the mixture is stirred for a period of: about 2 hours to about 72 hours, about 5 hours to about 8 to about 36 hours, about 12 to about 30 hours, or about 14 to about 28 hours.

52. The process according to claim 38, wherein the Tafamidis Form Va is isolated using at least one of filtration, centrifugation, or decantation.

53. The process according to claim 38, further comprising combining the product with at least one pharmaceutically acceptable excipient to provide a pharmaceutical formulation, including at least one of a tablet or capsule.

54-58. (canceled)

59. A pharmaceutical composition comprising a product according to claim 1, and at least one pharmaceutically acceptable excipient.

60. The pharmaceutical composition according to claim 59, wherein the at least one pharmaceutically acceptable excipient is at least one of a solubilizer, a surfactant, or an emulsifier.

61. The pharmaceutical composition according to claim 59, wherein the pharmaceutical excipient comprises a polyethylene glycol including at least one of PEG-400, PEG-300, or PEG-200.

62. The pharmaceutical composition according to claim 59, wherein the pharmaceutically acceptable excipient comprises at least one non-ionic surfactant including at least one of a hydrophilic non-ionic surfactant, an ethoxylated sorbitan ester, a polyoxyethylene sorbitan ester, or a polysorbate selected from one of polysorbate 20, polysorbate 60, or polysorbate 80.

63. The pharmaceutical composition according to claim 59, wherein the pharmaceutically acceptable excipient comprises a non-ionic emulsifier including at least one of a lipophilic non-ionic emulsifier, a sorbitan fatty acid ester, sorbitan monostearate, or sorbitan monooleate.

64. The pharmaceutical composition according to claim 59, wherein the pharmaceutically acceptable excipient comprises a polyvinylpyrrolidone having a molecular weight of between about 45 kDa to about 3000 kDa, about 60 kDa to about 2000 kDa, about 80 kDa to about 180 kDa, or about 100 kDa to about 150 kDa.

65. (canceled)

66. A process for preparing a pharmaceutical composition, comprising combining a crystalline product according to claim 1 with at least one pharmaceutically acceptable excipient.

67. (canceled)

68. (canceled)

69. A crystalline product according to claim 1, for use in the treatment of at least one of transthyretin-mediated amyloidosis familial amyloid polyneuropathy, cardiomyopathy or wild type or hereditary transthyretin-mediated amyloidosis, or transthyretin amyloidosis in adult patients with stage 1 symptomatic polyneuropathy.

70. A method of treating at least one of transthyretin-mediated amyloidosis, familial amyloid polyneuropathy, cardiomyopathy or wild type or hereditary transthyretin-mediated amyloidosis, or transthyretin amyloidosis in adult patients with stage 1 symptomatic polyneuropathy, the method comprising administering a therapeutically effective amount of a crystalline product according to claim 1, to a subject in need of the treatment.

71. (canceled)

72. A process for preparing a solid state form of tafamidis, tafamidis solvate, or tafamidis salt, comprising preparing a crystalline product according to claim 1, and converting it to another a solid state form thereof.
